(19) United States
(12) Plant Patent Application Publication
(10) Pub. No.: US 2003/0204891 P1
(43) Pub. Date: Oct. 30, 2003

(54) Fuchsia plant named 'Sanisutanfu akw3'

(76) Inventors: Toshi Tanaka, Ashiya-shi (JP); Hidefumi Funakoshi, Nishinomiya-shi (JP); Mito Ikeda, Nishinomiya-shi (JP); Kenkichi Nose, Kobe-shi (JP); Ushio Sakazaki, Hikone-shi (JP)

Correspondence Address: BURNS, DOANE, SWECKER & MATHIS, L.L.P., P.O. Box 1404, Alexandria, VA, 22313-1404, US

(21) Appl. No.: 10/132,477

(22) Filed: Apr. 26, 2002

(51) Int. Cl. A01H 5/00

(52) U.S. Cl. PLT/300

(57) ABSTRACT

Disclosed herein is a fuchsia plant named ‘SANISUTANFU akw3’ having a spreading habit, short internode and plentiful blooming. Leaf shape is ovate. Leaf arrangement is opposite, whorled with three leaves on flowering branches. Immature leaf color is grayish green, mature leaf color is moderate olive green (R.H.S.C.C. No.146A) without blotches. Petiol color is purplish red. Flower type is single and small. Petal shape is ovate and petal form is cup shape. The color of petal is pinkish white (R.H.S.C.C. No.56D) with vivid red (R.H.S.C.C. No.57B) variegation on it. The sepal does not twist and is slightly bending outside, with no variegation. The sepal is vivid pink (R.H.S.C.C. No.52A) and having oblong shape. The calyx tube is bell shape, being thin, short and vivid pink (R.H.S.C.C. No.52A) without variegation. The plant has high resistance to cold and heat, and can survive summer and frosty seasons in western Japan. Flowering season starts from the end of May, it is later in this group, into the end of autumn.

### BACKGROUND OF THE VARIETY

The present invention relates to a new and distinct variety of fuchsia plant obtained from a crossing ‘SANISTANF’ as female parent and a seedling of F1 of ‘Snow cap’ (Fuchsia×hyb.) as pollen parent.

The fuchsia plant is a very popular plant in the United States and Europe and is used for potting in the spring season to summer season. But the fuchsia plant have a weak resistance to heat and it is necessary for growing in air-conditioned room in term of intensity of heat. Especially in Japanese' summer, the heat is intense and it is necessary that the fuchsia plants grow in air-conditioned room, thus the fuchsia plants were not become popular. Accordingly, this invention was aimed at obtaining a new variety having strong resistance to heat and spreading growth habit with fine branching.

The new variety of fuchsia plant according to this invention originated from a crossing of ‘SANISTANF’ as female parent and a seedling of F1 of ‘Snow cap’ (Fuchsia×hyb.) as pollen parent.

First of all, in February 1991, a wild type Fuchsia native to Brazil was introduced, and a seedling that had vivid red sepal, dark bluish purple petals and high heat resistance was selected as pollen parent. And then controlled crossing was conducted with that seedling as a pollen parent and ‘Snow cap’ (Fuchsia×hyb.) as a female parent from June 1991. From that crossing, seedlings were obtained and the heat resistance of them was examined in 1994, and one seedling that had white petals was selected. From 1995, controlled crossing was conducted with that selected seedling as the pollen parent and ‘SANISTANF’ as the female parent. From that crossing, 88 seedlings were obtained. From 1996, the botanical characteristics and the heat resistance of those seedlings were examined. 10 seedlings were selected which were distinguishable from the control variety and stable in its characteristics. Then the selected plants were propagated by the use of cuttings, and have been grown as trial by flower potting. The botanical characteristics of the plants were examined. As a result, one seedling was selected and it was concluded that this new variety is uniform, stable in its characteristics, distinguishable from any other variety. And then this variety of fuchsia plant was named ‘SANISUTANFU akw3’.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). The color chart based on The Japan Color Standard for Horticultural Plant (J.H.S. Color Chart) is also added for reference.

This wild type of fuchsia plant and a seedling of F1 of ‘Snow cap’ (Fuchsia×hyb.) are presently maintained at Oumi Nursery Center of SUNTORY Ltd. at 863-1, Aza-Iketani, Oomori-cho, Yokaichi-shi, Shiga-ken, Japan.

This new variety of fuchsia plant named ‘SANISTUANFU akw3’ was asexually reproduced by cutting at the Oumi Nursery Center of SUNTORY Ltd. at 863-1, Aza-Iketani, Oomori-cho, Yokaichi-shi, Shiga-ken, Japan and the homogeneity and stability thereof were confirmed.

The female plant used in the crossing for ‘SANISUTANFU akw3’ and used as the comparison variety for the new variety ‘SANISUTANFU akw3’ was ‘SANISTANF’, which was registered in US (U.S. Plant Pat. No. 10,377). The botanical characteristics of the female plant ‘SANISTANF’ are as follows:

Plant:

Growth habit.—Spreading.

Plant height.—Medium.

Grade of bushiness.—Many stems.

Stem:

Thickness.—2.9 mm.

Color.—Yellow green.

Pubescence.—Sparse.

Branching.—Medium.

Length of internode.—Medium.

Leaf:

Phyllotaxis.—Opposite, partially 3-verticillate.

Shape.—Ovate.

Tip form of leaf.—Acuminate.

Base form of leaf.—Orbicular.

Shape of margin.—Dentate serrate.

Length.—47.9 mm.

Width.—28.2 mm.

Thickness.—0.4 mm.

Color of upper surface of new leaf.—Moderate yellow green. (R.H.S. 146B, J.H.S. 3514).

Color of upper surface of mature leaf.—Grayish olive green. (R.H.S. 137A, J.H.S. 3716).

Color of under surface of mature leaf.—Moderate yellow green (R.H.S. 146B, J.H.S. 3514) with dark red. (R.H.S. 59A, J.H.S. 0110) dots.

Variegation on upper surface.—Absent.

Color of leaf vein.—Dark red. (R.H.S. 59A, J.H.S. 0110).

Luster.—Absent.

Pubescence of upper surface.—Absent.

Pubescence of under surface.—Absent.

Length of petiole.—12.5 mm.

Width of petiole.—1.1 mm.

Color of petiole.—Dark red. (R.H.S. 59A, J.H.S. 0110).

Pubescence of petiole.—Sparse.

Flower:

Inflorescence types.—Leaf axil — single.

Facing direction.—Downward to horizontal.

Type.—Single.

Diameter.—43.4 mm.

Length.—33.1 mm.

Diameter of corolla.—Medium.

Shape of petal.—Obovate.

Length of petal.—16.2 mm.

Width of petal.—19.9 mm.

Thickness of petal.—0.4 mm.

Number of petals.—Medium: 4.

Color of petal at the beginning of blooming time.—Deep violet. (R.H.S. 93A, J.H.S. 8006).

Color of petal at blooming time.—Moderate purple. (R.H.S. 83B, J.H.S. 8613).

Variegated pattern of petal.—Absent.

Shape of sepal.—Oblong.

Tip form of sepal.—Acute.

Calyx rolling.—Medium (half up).

Intorsion of sepal.—None.

Curvature of sepal.—Outcurve.

Length of sepal.—28.8 mm.

Width of sepal.—11.0 mm.

Thickness of sepal.—0.7 mm.

Number of sepals.—Medium; 4.

Color of sepal.—Vivid red. (R.H.S. 62A, J.H.S. 0106).

Variegated pattern on sepal.—Absent.

Shape of calyx tube.—Long-tubular.

Thickness of calyx tube.—5.0 mm.

Length of calyx tube.—14.3 mm.

Color of calyx tube.—Vivid red. (R.H.S. 52A, J.H.S 0106).

Variegated pattern on calyx tube.—Absent.

Length of pistil.—Long.

Color of pistil.—Deep purplish pink. (R.H.S. 55A, J.H.S. 9705).

Color of stigma.—Moderate purplish red. (R.H.S. 58A, J.H.S. 9714).

Length of stamen.—Long.

Color of stamen.—Strong purplish red. (R.H.S. 60D, J.H.S. 9708).

Number of stamens.—Medium; 8.

Thickness of peduncle.—Medium.

Length of peduncle.—Medium.

Color of peduncle.—Pale yellowish pink. (R.H.S. 37D, J.H.S. 1002).

Shape of ovary.—Oblong.

Length of ovary.—Medium.

Color of ovary.—Light yellow green. (R.H.S. 144D, J.H.S. 3503).

Number of flowers.—Medium.

Flower fragrance.—Absent.

Floriferousness.—Numerous flowers.

Grade of seed fertility.—Medium.

Grade of floral abscission.—Medium.

Blooming time.—Early.

Flowering duration.—Long: early May to the middle of December.

Physiological and ecological characteristics:

Grade of cold resistance.—Strong.

Grade of heat resistance.—Strong.

Grade of disease resistance.—Strong.

Grade of pest resistance.—Medium.

### SUMMARY OF THE VARIETY

This new variety of fuchsia plant named ‘SANISUTANFU akw3’ has a spreading growth habit, short internode and plentiful blooming. Flower type is single and facing direction is arching downward. Flower is small and petal shape is ovate. The color of petal is pinkish white (R.H.S. C.C. No.56D) with vivid red (R.H.S. C.C. No.57B) variegation on it. Shape of calyx tube is bell shape and color of calyx tube is vivid pink (R.H.S. C.C. No.52A). Stamen color is pink. The plant has high resistance to cold and heat, and can survive summer and frosty seasons in western Japan. Flowering season starts from the end of May, it is later in this group, into the end of autumn.

### BRIEF DESCRIPTION OF THE DRAWINGS

FIG. 1 is photograph giving a partial view of the new variety of fuchsia plant named ‘SANISUTANFU akw3’.

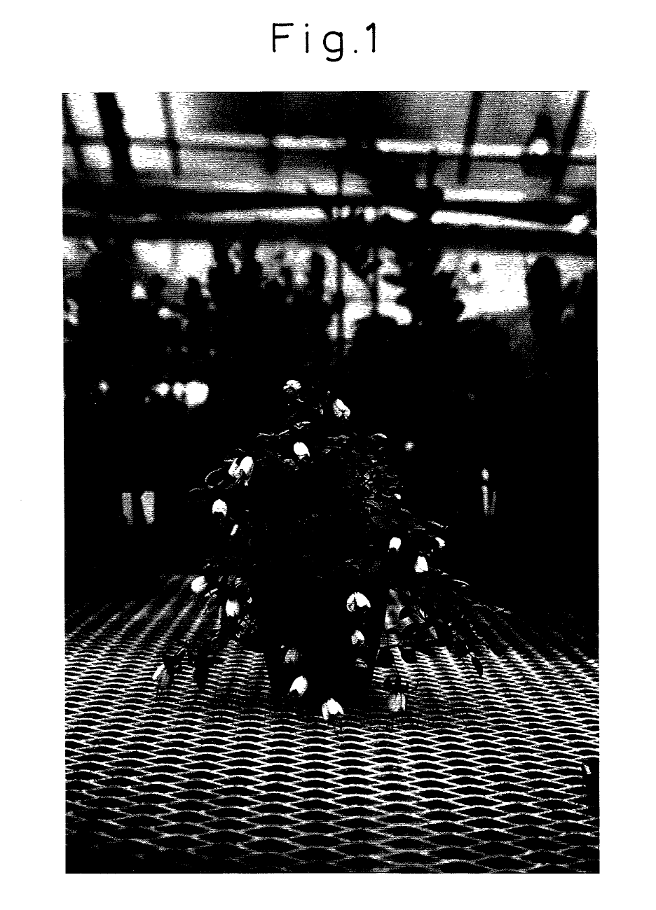

FIG. 2 is a photograph of flowers of the new variety of fuchsia plant named ‘SANISUTANFU akw3’.

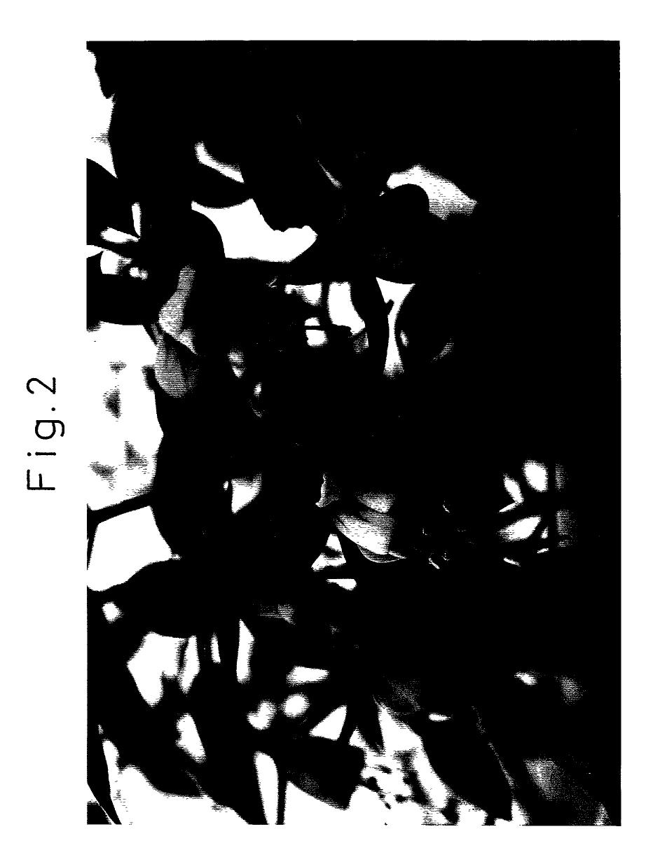

### DESCRIPTION OF THE VARIETY

The botanical characteristics of the new and distinct variety of fuchsia plant ‘SANISUTANFU akw3’ are as follows. This investigation was held at Nishinomiya-shi, Hyogo, Japan. Plant materials, propagated by stem cuttings, examined in this investigation had been cultivated from the spring in 1995 to the autumn in 1997.

Plant:

Growth habit.—Spreading.

Plant height.—Medium.

Grade of bushiness.—Medium stems.

Stem:

Thickness.—2.4 mm.

Color.—Purplish red.

Pubescence.—Medium.

Branching.—Medium.

Length of internode.—Short (2.9 cm).

Leaf:

Phyllotaxis.—Opposite, partially 3-verticillate.

Shape.—Ovate.

Tip form of leaf.—Acute.

Base form of leaf.—Orbicular.

Shape of margin.—Dentate serrate.

Length.—4.8 cm.

Width.—2.6 cm.

Thickness.—0.3 mm.

Color of upper surface of new leaf.—Grayish green.

Color of upper surface of mature leaf.—Moderate olive green. (R.H.S. 146A, J.H.S. 3508).

Color of under surface of mature leaf.—Moderate yellow green (R.H.S. 146B, J.H.S. 3514) without blotches.

Variegation on upper surface.—Absent.

Color of leaf vein.—Dark red. (R.H.S. 59A, J.H.S. 0110).

Luster.—Absent.

Pubescence of upper surface.—Absent.

Pubescence of under surface.—Absent.

Length of petiole.—1.3 cm.

Width of petiole.—0.9 mm.

Color of petiole.—Dark red. (R.H.S. 59A, J.H.S. 0110).

Pubescence of petiole.—Medium.

Flower:

Inflorescence types.—Leaf axil — single.

Facing direction.—Downward to horizontal.

Type.—Single.

Diameter.—4.4 cm.

Length.—2.8 cm.

Diameter of corolla.—Small (1.5 cm).

Shape of petal.—Obovate.

Length of petal.—1.5 cm.

Width of petal.—1.6 cm.

Thickness of petal.—0.3 mm.

Number of petals.—Many: 5-7.

Color of petal at the beginning of blooming time.—Pinkish white. (R.H.S. 56D, J.H.S. 0401) with vivid red (R.H.S. 57B, J.H.S. 0107).

Color of petal at blooming time.—Pinkish white. (R.H.S. 56D, J.H.S. 0401) with vivid red (R.H.S. 57B, J.H.S. 0107).

Variegated pattern of petal.—Present.

Shape of sepal.—Oblong.

Tip form of sepal.—Acuminate.

Calyx rolling.—Slightly.

Intorsion of sepal.—None.

Curvature of sepal.—Flat.

Length of sepal.—2.1 cm.

Width of sepal.—0.8 cm.

Thickness of sepal.—0.7 mm.

Number of sepals.—Medium; 4.

Color of sepal.—Vivid red. (R.H.S. 62A, J.H.S. 0106).

Variegated pattern on sepal.—Absent.

Shape of calyx tube.—Long-tubular.

Thickness of calyx tube.—4.0 mm.

Length of calyx tube.—0.5 cm.

Color of calyx tube.—Vivid red. (R.H.S. 52A, J.H.S 0106).

variegated pattern on calyx tube.—Absent.

Length of pistil.—Short (4.7 cm).

Color of pistil.—Vivid pink. (R.H.S. 52A).

Color of stigma.—Moderate purplish red. (R.H.S. 58A, J.H.S. 9714).

Length of stamen.—Medium (2.5 cm).

Color of stamen.—Pink.

Number of stamens.—Medium; 8.

Thickness of peduncle.—Medium (0.7 mm).

Length of peduncle.—Medium (2.6 cm).

Color of peduncle.—Yellowish green.

Shape of ovary.—Oblong.

Length of ovary.—Short (0.6 cm).

Color of ovary.—Light yellow green. (R.H.S. 144D, J.H.S. 3503).

Number of flowers.—Many.

Flower fragrance.—Absent.

Floriferousness.—Numerous flowers.

Grade of seed fertility.—Medium.

Grade of floral abscission.—Difficult.

Blooming time.—Early.

Flowering duration.—Long: Early May to the middle of December.

Physiological and ecological characteristics:

Grade of cold resistance.—Strong.

Grade of heat resistance.—Strong.

Grade of disease resistance.—Medium.

Grade of pest resistance.—Medium.

This new variety ‘SANISUTANFU akw3’ was asexually reproduced by cutting.

The plant of this new variety ‘SANISUTANFU akw3’ is presently planted and maintained at the Omi Research Center of SUNTORY Ltd.

1. A new and distinct variety of fuchsia plant named ‘SANISUTANFU akw3’, substantially as herein illustrated and described, characterized particularly as to novelty by (A) being a spreading growth habit having short internodes, (B) a great profusion of blooms, the whole bush remaining in bloom for considerable period of time, (C) flower is single and the petal color is pinkish white with vivid red variegation on it, sepal color is vivid red and (D) having a high tolerance to heat and cold.
